(19) United States
(12) Patent Application Publication
(10) Pub. No.: US 2021/0000326 A1
(43) Pub. Date: Jan. 7, 2021

(54) ENDOSCOPIC IMAGE PROCESSING APPARATUS, ENDOSCOPIC IMAGE PROCESSING METHOD, AND RECORDING MEDIUM

(71) Applicant: OLYMPUS CORPORATION, Tokyo (JP)

(72) Inventors: Hiromu SUGITA, Tokyo (JP); Yamato KANDA, Tokyo (JP); Katsuyoshi TANIGUCHI, Tokyo (JP); Makoto KITAMURA, Tokyo (JP)

(73) Assignee: OLYMPUS CORPORATION, Tokyo (JP)

(21) Appl. No.: 16/934,629

(22) Filed: Jul. 21, 2020

Related U.S. Application Data
(63) Continuation of application No. PCT/JP2018/002503, filed on Jan. 26, 2018.

(51) Int. Cl. A61B 1/00; G06T 7/00

(52) U.S. Cl. CPC A61B 1/0005; A61B 1/00004; G06T 2207/30096; G06T 2207/10068; G06T 7/0012

(57) ABSTRACT

An endoscopic image processing apparatus includes a processor. The processor detects a lesion candidate region included in an endoscopic image obtained by picking up an image of an inside of a subject with an endoscope, highlights a position of the lesion candidate region detected from the endoscopic image, when a plurality of lesion candidate regions are detected from the endoscopic image, relatively evaluates visibility of the plurality of lesion candidate regions, and performs setting for position highlighting of the lesion candidate region based on an evaluation result of the visibility.

### CROSS REFERENCE TO RELATED APPLICATION

This application is a continuation application of PCT/JP2018/002503 filed on Jan. 26, 2018, the entire contents of which are incorporated herein by this reference.

### BACKGROUND OF THE INVENTION

### 1. Field of the Invention

The present invention relates to an endoscopic image processing apparatus, an endoscopic image processing method, and a recording medium.

2. Description of the Related Art

In endoscopic observation in a medical field, there has been known a technique for detecting a lesion candidate region from an endoscopic image obtained by picking up an image of a desired part in a subject, adding visual information for informing presence of the detected lesion candidate region to the endoscopic image, and displaying the visual information.

More specifically, for example, International Publication No. 2017/073338 discloses a technique for detecting a lesion candidate region from an observation image obtained by picking up an image of an inside of a subject with an endoscope and displaying a display image obtained by adding a marker image surrounding the detected lesion candidate region to the observation image.

### SUMMARY OF THE INVENTION

An endoscopic image processing apparatus according to an aspect of the present invention includes a processor. The processor detects a lesion candidate region included in an endoscopic image obtained by picking up an image of an inside of a subject with an endoscope, highlights a position of the lesion candidate region detected from the endoscopic image, when a plurality of lesion candidate regions are detected from the endoscopic image, relatively evaluates visibility of the plurality of lesion candidate regions, and performs setting for position highlighting of the lesion candidate region based on an evaluation result of the visibility.

An endoscopic image processing method according to an aspect of the present invention includes: detecting a lesion candidate region included in an endoscopic image obtained by picking up an image of an inside of a subject with an endoscope; highlighting a position of the lesion candidate region detected from the endoscopic image; when a plurality of lesion candidate regions are detected from the endoscopic image, relatively evaluating visibility of the plurality of lesion candidate regions; and performing setting for position highlighting, of the lesion candidate region based on an evaluation result of the visibility.

A recording medium according to an aspect of the present invention is a computer-readable non-transitory recording medium that stores a program, the program causing a computer to: detect a lesion candidate region included in an endoscopic image obtained by picking up an image of an inside of a subject with an endoscope; highlight a position of the lesion candidate region detected from the endoscopic image; when a plurality of lesion candidate regions are detected from the endoscopic image, relatively evaluate visibility of the plurality of lesion candidate regions; and perform setting for position highlighting of the lesion candidate region based on an evaluation result of the visibility.

### BRIEF DESCRIPTION OF THE DRAWINGS

FIG. 1 is a diagram showing a configuration of a main part of an endoscope system including an endoscopic image processing apparatus according to an embodiment;

FIG. 2 is a flowchart for explaining a specific example of processing performed in the endoscopic image processing apparatus according to a first embodiment;

FIG. 3 is a diagram schematically showing an example of an endoscopic image set as a processing target of the endoscopic image processing apparatus according to the first embodiment;

FIG. 4 is a diagram for explaining a specific example of processing performed on the endoscopic image shown in FIG. 3;

FIG. 5 is a diagram schematically showing an example of a display image displayed on a display apparatus through processing of the endoscopic image processing apparatus according to the first embodiment;

FIG. 6 is a flowchart for explaining a specific example of processing performed in an endoscopic image processing apparatus according to a second embodiment;

FIG. 7 is a diagram schematically showing an example of an endoscopic image set as a processing target of the endoscopic image processing apparatus according to the second embodiment;

FIG. 8 is a diagram schematically showing an example of a display image displayed on a display apparatus through processing of the endoscopic image processing apparatus according to the second embodiment;

FIG. 9 is a flowchart for explaining a specific example of processing performed in an endoscopic image processing apparatus according to a third embodiment;

FIG. 10 is a diagram schematically showing an example of an endoscopic image set as a processing target of the endoscopic image processing apparatus according to the third embodiment; and

FIG. 11 is a diagram schematically showing an example of a display image displayed on a display apparatus through processing of the endoscopic image processing apparatus according to the third embodiment.

### DETAILED DESCRIPTION OF THE PREFERRED EMBODIMENTS

Embodiments of the present invention are explained below with reference to the drawings.

### (First Embodiment)

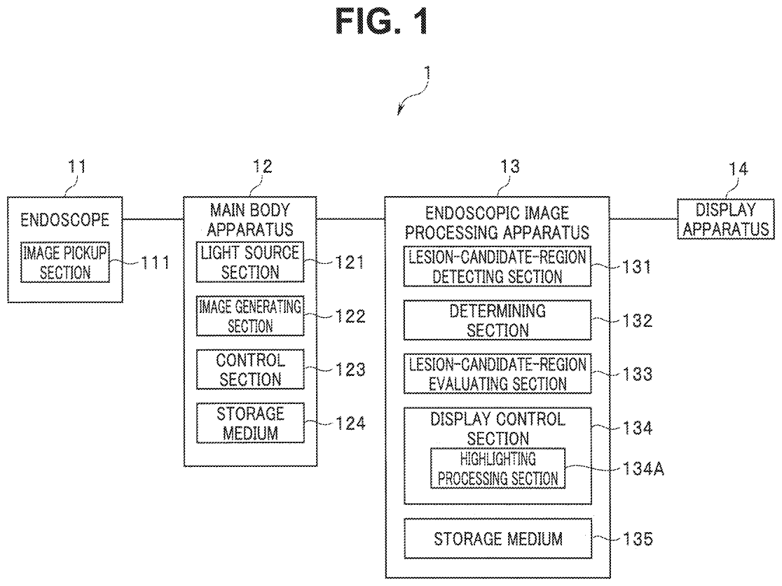

FIGS. 1 to 5 relate to a first embodiment of the present invention.

An endoscope system 1 includes, as shown in FIG. 1, an endoscope 11, a main body apparatus 12, an endoscopic image processing apparatus 13, and a display apparatus 14. FIG. 1 is a diagram showing a configuration of a main part of an endoscope system including an endoscopic image processing apparatus according to the embodiment.

The endoscope 11 includes, for example, an elongated insertion section (not illustrated) insertable into a subject and an operation section (not illustrated) provided at a proximal end portion of the insertion section. For example, the endoscope 11 is detachably connected to the main body apparatus 12 via a universal cable (not illustrated) extending from the operation section. A light guide member (not illustrated) such as an optical fiber for guiding illumination light supplied from the main body apparatus 12 and emitting the illumination light from a distal end portion of the insertion section is provided on an inside of the endoscope 11. An image pickup section 111 is provided at the distal end of the insertion section of the endoscope 11

The image pickup section 111 includes, for example, a CCD image sensor or a CMOS image sensor. The image pickup section 111 is configured to pick up an image of return light from an object illuminated by the illumination light emitted through the distal end portion of the insertion section, generate an image pickup signal corresponding to the return light, the image of which is picked up, and output the image pickup signal to the main body apparatus 12.

The main body apparatus 12 is detachably connected to each of the endoscope 11 and the endoscopic image processing apparatus 13. The main body apparatus 12 includes, for example, as shown in FIG. 1, a light source section 121, an image generating section 122, a control section 123, and a storage medium 124.

The light source section 121 includes one or more light emitting elements such as LEDs. More specifically, the light source section 121 includes, for example, a blue LED that generates blue light, a green LED that generates green light, and a red LED that generates red light. The light source section 121 is configured to be able to generate illumination light corresponding to control by the control section 123 and supply the illumination light to the endoscope 11.

The image generating section 122 is configured to be able to generate an endoscopic image based on an image pickup signal outputted from the endoscope 11 and sequentially output the generated endoscopic image to the endoscopic image processing apparatus 13 frame by frame.

The control section 123 is configured to perform control relating to operation of sections of the endoscope 11 and the main body apparatus 12.

In the present embodiment, the image generating section 122 and the control section 123 of the main body apparatus 12 may be configured as individual electronic circuits or may be configured as circuit blocks in an integrated circuit such as an FPGA (field programmable gate array). In the present embodiment, for example, the main body apparatus 12 may include one or more CPUs. By modifying a configuration according to the present embodiment as appropriate, for example, the main body apparatus 12 may read, from the storage medium 124 such as a memory, a program for causing functions of the image generating section 122 and the control section 123 to be executed and may perform operation corresponding to the read program.

The endoscopic image processing apparatus 13 is detachably connected to each of the main body apparatus 12 and the display apparatus 14. The endoscopic image processing apparatus 13 includes a lesion-candidate-region detecting section 131, a determining section 132, a lesion-candidate-region evaluating section 133, a display control section 134, and a storage medium 135.

The lesion-candidate-region detecting section 131 is configured to perform processing for detecting a lesion candidate region L included in endoscopic images sequentially outputted from the main body apparatus 12 and perform processing for acquiring lesion candidate information IL, which is information indicating the detected lesion candidate region L. In other words, endoscopic images obtained by picking up an image of an inside of a subject with an endoscope are sequentially inputted to the lesion-candidate-region detecting section 131. The lesion-candidate-region detecting section 131 is configured to perform processing for detecting the lesion candidate region L included in the endoscopic images.

Note that, in the present embodiment, the lesion candidate region L is detected as, for example, a region including abnormal findings such as a polyp, bleeding, and a blood vessel abnormality. In the present embodiment, the lesion candidate information IL is acquired as, for example, information including position information indicating a position (a pixel position) of the lesion candidate region L included in an endoscopic image outputted from the main body apparatus 12 and size information indicating a size (the number of pixels) of the lesion candidate region L included in the endoscopic image.

In the present embodiment, for example, the lesion-candidate-region detecting section 131 may be configured to detect the lesion candidate region L based on a predetermined feature value obtained from an endoscopic image obtained by picking up an image of an inside of a subject with an endoscope or may be configured to detect the lesion candidate region L using a discriminator that has acquired, in advance, with a learning method such as deep learning, a function capable of discriminating an abnormal finding included in the endoscopic image.

The determining section 132 is configured to perform processing for determining, based on a processing result of the lesion-candidate-region detecting section 131, whether a plurality of lesion candidate regions L are detected from an endoscopic image for one frame.

The lesion-candidate-region evaluating section 133 is configured to, when a determination result indicating that a plurality of lesion candidate regions L are detected from the endoscopic image for one frame is obtained by the determining section 132, perform processing for evaluating states of the plurality of lesion candidate regions L included in the endoscopic image for one frame. Note that a specific example of the processing performed in the lesion-candidate-region evaluating section 133 is explained below.

The display control section 134 is configured to perform processing for generating a display image using the endoscopic images sequentially outputted from the main body apparatus 12 and perform processing for causing the display apparatus 14 to display the generated display image. The display control section 134 includes a highlighting processing section 1341 that performs highlighting processing for highlighting the lesion candidate region L detected from the endoscopic image by the processing of the lesion-candidate-region detecting section 131. The display control section 134 is configured to perform processing for setting, based on the determination result of the determining section 132 and an evaluation result of the lesion-candidate-region evaluating section 133, a marker image M (explained below) added by highlighting processing of the highlighting processing section 134A. In other words, the display control section 134 has a function of a highlighting-processing setting section and is configured to, when the determination result indicating that a plurality of lesion candidate regions L are detected from the endoscopic image for one frame is obtained by the determining section 132, perform, based on the evaluation result of the lesion-candidate-region evaluating section 133, setting for processing performed in the highlighting processing section 134A.

The highlighting processing section 134A is configured to generate, based on the lesion candidate information IL acquired by the lesion-candidate-region detecting section 131, the marker image M for highlighting a position of the lesion candidate region L detected from the endoscopic image by the processing of the lesion-candidate-region detecting section 131 and perform, as the highlighting processing, processing for adding the generated marker image M to the endoscopic image. Note that, as long as the highlighting processing section 134A generates the marker image M for highlighting the position of the lesion candidate region L, the highlighting processing section 134A may perform the highlighting processing using only the position information included in the lesion candidate information IL or may perform the highlighting processing using both of the position information and the size information included in the lesion candidate information IL.

In the present embodiment, the endoscopic image processing apparatus 13 includes a processor. Sections of the processor may be configured as individual electronic circuits or may be configured as circuit blocks in an integrated circuit such as an FPGA (field programmable gate array). In the present embodiment, for example, the processor of the endoscopic image processing apparatus 13 may include one or more CPUs. By modifying the configuration according to the present embodiment as appropriate, for example, the processor of the endoscopic image processing apparatus 13 may read, from the storage medium 135 such as a memory, a program for causing functions of the lesion-candidate-region detecting section 131, the determining section 132, the lesion-candidate-region evaluating section 133, and the display control section 134 to be executed and may perform operation corresponding to the read program. By modifying the configuration according to the present embodiment as appropriate, for example, the functions of the sections of the endoscopic image processing apparatus 13 may be incorporated as functions of the main body apparatus 12.

The display apparatus 14 includes a monitor or the like and is configured to be able to display a display image outputted through the endoscopic image processing apparatus 13.

Subsequently, action of the present embodiment is explained. Note that, in the following explanation, an example is explained in which blue light, green light, and red light are sequentially or simultaneously emitted from the light source section 121 as illumination light corresponding to the control by the control section 123, that is, an endoscopic image including color components of blue, green, and red is generated by the image generating section 122.

After connecting the sections of the endoscope system 1 and turning on a power supply, the user such as a surgeon inserts the insertion section of the endoscope 11 into an inside of a subject and arranges the distal end of the insertion section in a position where an image of a desired observation part on the inside of the subject can be picked up. According to such operation by the user, illumination light is supplied from the light source section 121 to the endoscope 11. An image of return light from the object illuminated by the illumination light is picked up in the image pickup section 111. An endoscopic image corresponding to an image pickup signal outputted from the image pickup section 111 is generated in the image generating section 122 and is outputted to the endoscopic image processing apparatus 13.

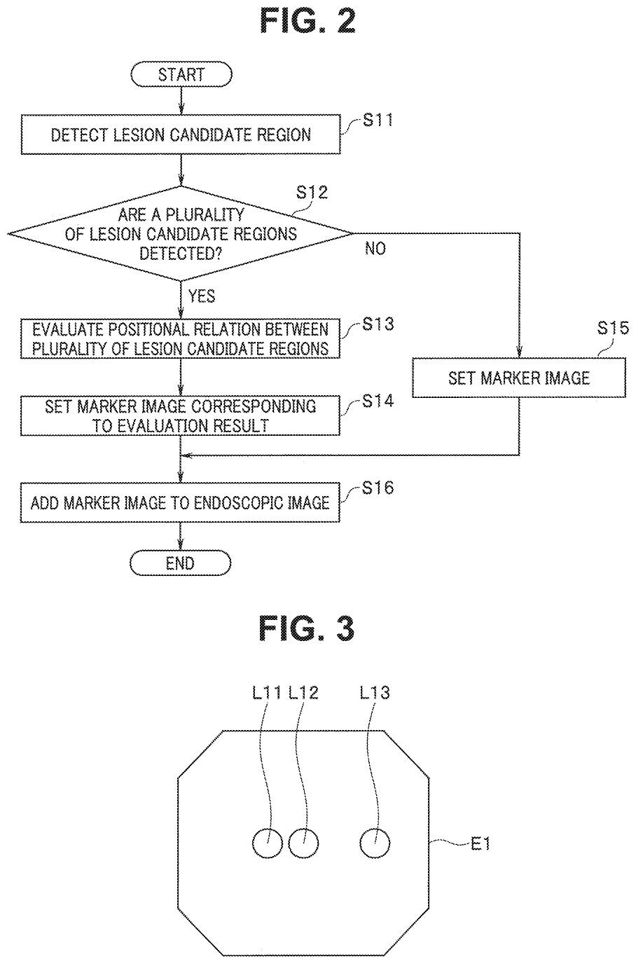

A specific example of processing performed in the sections of the endoscopic image processing apparatus 13 in the present embodiment is explained with reference to FIG. 2 and the like. Note that, in the following explanation, it is assumed that one or more lesion candidate regions L are included in an endoscopic image outputted from the main body apparatus 12. FIG. 2 is a flowchart for explaining a specific example of processing performed in the endoscopic image processing apparatus according to the first embodiment.

The lesion-candidate-region detecting section 131 performs processing for detecting the lesion candidate region L included in the endoscopic image outputted from the main body apparatus 12 and performs processing for acquiring the lesion candidate information IL, which is information indicating the detected lesion candidate region L (step S11 in FIG. 2).

More specifically, according to the processing in step S11 in FIG. 2, for example, the lesion candidate-region detecting section 131 detects three lesion candidate regions L11, L12, and L13 included in an endoscopic image E1 for one frame shown in FIG. 3 and respectively acquires lesion candidate information IL11 corresponding to the lesion candidate region L11, lesion candidate information IL12 corresponding to the lesion candidate region L12, and lesion candidate information IL13 corresponding to the lesion candidate region L13. In other words, in such a case, the lesion candidate regions L11, L12, and L13 and the lesion candidate information IL11, IL12, and IL13 are acquired as a processing result of step S11 in FIG. 2. FIG. 3 is a diagram schematically showing an example of an endoscopic image set as a processing target of the endoscopic image processing; apparatus according to the first embodiment.

The determining section 132 performs processing for determining, based on the processing result of step S11 in FIG. 2, whether a plurality of lesion candidate regions L are detected from an endoscopic image for one frame (step S12 in FIG. 2).

When a determination result indicating that a plurality of lesion candidate regions L are detected from the endoscopic image for one frame is obtained (S12: YES), the lesion-candidate-region evaluating section 133 performs processing for evaluating a positional relation between the plurality of lesion candidate regions L included in the endoscopic image for one frame (step S13 in FIG. 2).

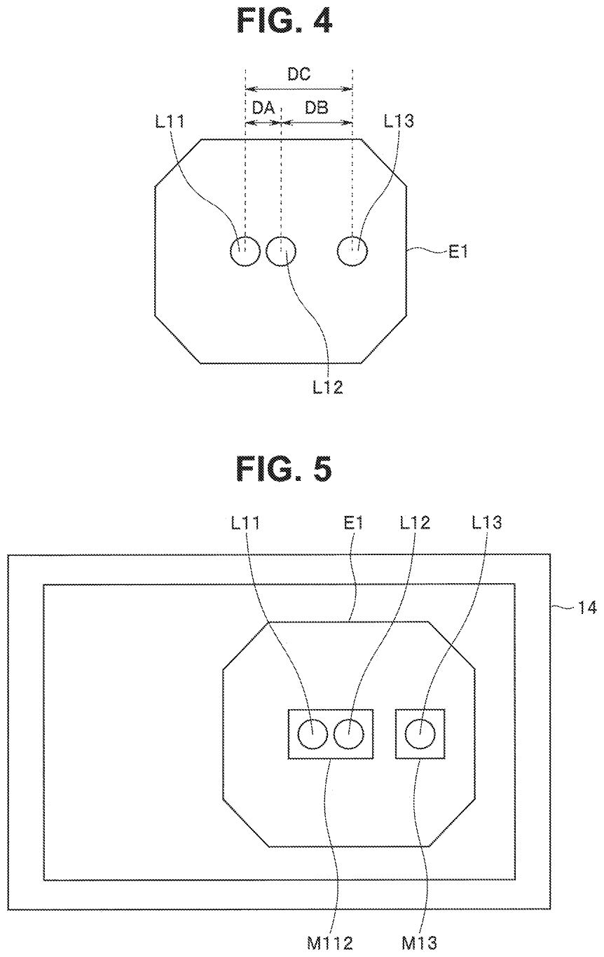

More specifically, for example, the lesion-candidate-region evaluating section 133 respectively calculates, based on the lesion candidate information IL11, IL12, and IL13, a relative distance DA equivalent to a distance between centers of the lesion candidate regions L11 and L12, a relative distance DB equivalent to a distance between centers of the lesion candidate regions L12 and L13, and a relative distance DC equivalent to a distance between centers of the lesion candidate regions L11 and L13 (see FIG. 4). FIG. 4 is a diagram for explaining a specific example of processing performed on the endoscopic image shown in FIG. 3.

For example, the lesion-candidate-region evaluating section 133 compares the relative distance DA and a predetermined threshold THA to thereby evaluate a positional relation between the lesion candidate regions L11 and L12. For example, when obtaining a comparison result indicating DA≤THA, the lesion-candidate-region evaluating section 133 obtains an evaluation result indicating that the lesion candidate regions L11 and L12 are present in positions close to each other. For example, when obtaining a comparison result indicating DA>THA, the lesion-candidate-region evaluating section 133 obtains an evaluation result indicating that the lesion candidate regions LII and L12 are present in positions far apart from each other. Note that, in FIG. 4, an example is shown in which DA≤THA, that is, the evaluation result indicating that the lesion candidate regions L11 and L12 are present in the positions close to each other is obtained.

For example, the lesion-candidate-region evaluating section 133 compares the relative distance DB and the predetermined threshold THA to thereby evaluate a positional relation between the lesion candidate regions L12 and L13. For example, when obtaining a comparison result indicating DB≤THA, the lesion-candidate-region evaluating section 133 obtains an evaluation result indicating that the lesion candidate regions L12 and L13 are present in positions close to each other. For example, when obtaining a comparison result indicating DB>THA, the lesion-candidate-region evaluating section 133 obtains an evaluation result indicating that the lesion candidate regions L12 and L13 are present in positions far apart from each other. Note that, in FIG. 4, an example is shown in which DB>THA, that is, the evaluation result indicating that the lesion candidate regions L12 and L13 are present in the positions far apart from each other is obtained.

For example, the lesion-candidate-region evaluating section 133 compares the relative distance DC and the predetermined threshold THA to thereby evaluate a positional relation between the lesion candidate regions L11 and L13. For example, when obtaining a comparison result indicating DC≤THA, the lesion-candidate-region evaluating section 133 obtains an evaluation result indicating that the lesion candidate regions L11 and L13 are present in positions close to each other. For example, when obtaining a comparison result indicating DC>THA, the lesion-candidate-region evaluating section 133 obtains an evaluation result indicating that the lesion candidate regions L11 and L13 are present in positions far apart from each other. Note that, in FIG. 4, an example is shown in which DC>THA, that is, the evaluation result indicating that the lesion candidate regions L11 and L13 are present in the positions far apart from each other is obtained.

When the determination result indicating that a plurality of lesion candidate regions L are detected from the endoscopic image for one frame is obtained (S12: YES), the display control section 134 performs processing for setting, based on the evaluation result in step S13 in FIG. 2, the marker image M added by the highlighting processing of the highlighting processing section 134A (step S14 in FIG. 2).

More specifically, based on the evaluation result in step S13 in FIG. 2, for example, the display control section 134 sets a marker image M112 for collectively highlighting the positions of the lesion candidate regions L11 and L12 present in the positions close to each other and sets a marker image M13 for individually highlighting the position of the lesion candidate region L13 present in the position far apart from both of the lesion candidate regions L11 and L12.

In other words, according to the processing in step S14 in FIG. 3, when an evaluation result indicating that two lesion candidate regions among a plurality of lesion candidate regions detected from an endoscopic image for one frame are present in positions close to each other is obtained by the lesion-candidate-region evaluating section 133, setting for collectively highlighting the positions of the two lesion candidate regions is performed by the display control section 134.

When a determination result indicating that one lesion candidate region L is detected from the endoscopic image for one frame is obtained (S12: NO), the display control section 134 sets the marker image M for highlighting a position of the one lesion candidate region L (step S15 in FIG. 2). Note that, in the present embodiment, for example, the marker image M same as the marker image M13 may be set by the processing in step S15 in FIG. 2.

The highlighting processing section 134A performs processing for generating, based on the lesion candidate information IL obtained as the processing result of step S11 in FIG. 2, the marker image M set through the processing in step S14 or step S15 in FIG. 2 and adding the generated marker image M to the endoscopic image (step 516 in FIG. 2).

More specifically, for example, the highlighting processing section 134A performs processing for generating, based on the lesion candidate information IL11, IL12, and IL13, the marker images M112 and M13 set through the processing in step S14 in FIG. 2, adding the generated marker image M112 to peripheries of the lesion candidate regions L11 and L12 in the endoscopic image E1, and adding the generated marker image M13 to a periphery of the lesion candidate region L13 in the endoscopic image E1. According to such processing of the highlighting processing section 134A, for example, a display image obtained by respectively adding the marker image M112, which is a rectangular frame surrounding the peripheries of the lesion candidate regions L11 and L12, and the marker image M13, which is a rectangular frame surrounding the periphery of the lesion candidate region L13, to the endoscopic image E1 is generated. The generated display image is displayed on the display apparatus 14 (see FIG. 5). FIG. 5 is a diagram schematically showing an example of a display image displayed on the display apparatus through processing of the endoscopic image processing apparatus according to the first embodiment.

For example, the highlighting processing section 134A performs processing for generating, based on the lesion candidate information IL obtained as the processing result of step S11 in FIG. 2, the marker image M set through the processing in step S15 in FIG. 2 and adding the generated marker image M to a periphery of one lesion candidate region L in the endoscopic image. According to such processing of the highlighting processing section 134A, for example, a display image obtained by adding the marker image M (same as the marker image M13) surrounding the periphery of the lesion candidate region L to the endoscopic image E1 is generated. The generated display image is displayed on the display apparatus 14 (not illustrated).

As explained above, according to the present embodiment, a marker image for collectively highlighting positions of a plurality of lesion candidate regions present in positions close to each other can be added to an endoscopic image. Therefore, according to the present embodiment, it is possible to, without obstructing visual recognition of a lesion candidate region included in the endoscopic image as much as possible, inform presence of the lesion candidate region.

Note that, according to the present embodiment, the processing performed in step S13 in FIG. 2 is not limited to the processing for evaluating a positional relation between two lesion candidate regions L included in an endoscopic image based on a relative distance between the two lesion candidate regions L. For example, processing for evaluating the positional relation between the two lesion candidate regions L based on predetermined reference positions in the respective two lesion candidate regions L such as pixel positions equivalent to centers or centers of gravity of the respective two lesion candidate regions L may be performed.

According to the present embodiment, the processing performed in step S13 in FIG. 2 is not limited to processing for calculating a distance between centers of two lesion candidate regions L included in an endoscopic image as a relative distance. For example, a shortest distance between end portions of the two lesion candidate regions L included in the endoscopic image may be calculated as the relative distance.

According to the present embodiment, the processing performed in step S13 in FIG. 2 is not limited to processing for calculating a relative distance between two lesion candidate regions L included in an endoscopic image as a two-dimensional distance. For example, processing for calculating the relative distance as a three-dimensional distance may be performed by using, as appropriate, for example, a method disclosed in Japanese Patent Application Laid-Open Publication No. 2013-255656. According to the processing for calculating the relative distance between the two lesion candidate regions L included in the endoscopic image as the three-dimensional image, for example, when a luminance difference between the two lesion candidate regions L is small, it is possible to obtain an evaluation result indicating that the two lesion candidate regions L are present in positions dose to each other. When the luminance difference between the two lesion candidate regions L is large, it is possible to obtain an evaluation result indicating that the two lesion candidate regions L are present in positions far apart from each other.

According to the present embodiment, as long as it is possible to collectively highlight positions of a plurality of lesion candidate regions present in positions close to each other, a frame having a shape different from a rectangular frame may be added to an endoscopic image as a marker image.

According to the present embodiment, for example, when a marker image for collectively highlighting positions of a plurality of lesion candidate regions is added to an endoscopic image, a character string or the like indicating the number of lesion candidate regions set as highlighting targets by the marker image may be caused to be displayed together with the endoscopic image. More specifically, for example, when the marker image M112 is added to the endoscopic image E1, a character string or the like indicating that the number of lesion candidate regions surrounded by the marker image M112 is two may be caused to be displayed together with the endoscopic image E1.

### (Second Embodiment)

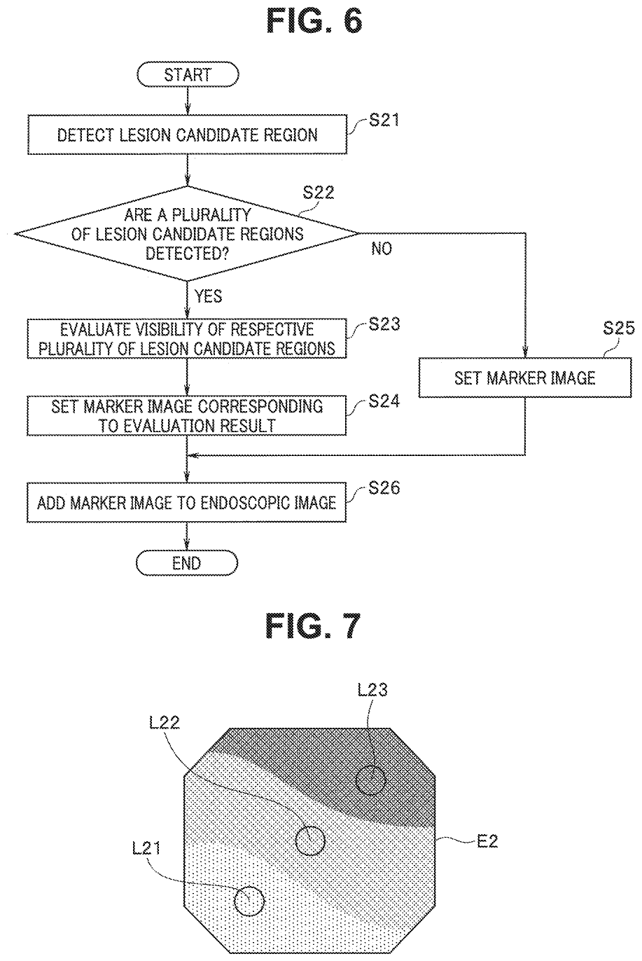

FIGS. 6 to 8 relate to a second embodiment of the present invention.

Note that, in the present embodiment, detailed explanation concerning portions having the same configurations and the like as the configurations and the like in the first embodiment is omitted. Portions having configurations and the like different from the configurations and the like in the first embodiment are mainly explained.

The endoscopic image processing apparatus 13 in the present embodiment is configured to perform processing different from the processing explained in the first embodiment. Specific examples of processing performed in sections of the endoscopic image processing apparatus 13 in the present embodiment are explained with reference to FIG. 6 and the like. FIG. 6 is a flowchart for explaining a specific example of processing performed in an endoscopic image processing apparatus according to the second embodiment.

The lesion-candidate-region detecting section 131 performs processing for detecting the lesion candidate region L included in an endoscopic image outputted from the main body apparatus 12 and performs processing for acquiring the lesion candidate information IL, which is information indicating the detected lesion candidate region L (step S21 in FIG. 6).

More specifically, according to the processing in step S21 in FIG. 6, the lesion-candidate-region detecting section 131 detects three lesion candidate regions L21, L22, and L23 included in an endoscopic image E2 for one frame shown in FIG. 7 and respectively acquires lesion candidate information IL21 corresponding to the lesion candidate region IL21, lesion candidate information IL22 corresponding to the lesion candidate region L22, and lesion candidate information IL23 corresponding to the lesion candidate region L23. In other words, in such a case, the lesion candidate regions L21, L22, and L23 and the lesion candidate information IL21, IL22, and IL23 are acquired as a processing result of step S21 in FIG. 6. FIG. 7 is a diagram schematically showing an example of an endoscopic image set as a processing target of the endoscopic image processing apparatus according to the second embodiment.

The determining section 132 performs processing for determining, based on the processing result of step S21 in FIG. 6, whether a plurality of lesion candidate regions L are detected from an endoscopic image for one frame (step S22 in FIG. 6).

When a determination result indicating that a plurality of lesion candidate regions L are detected from the endoscopic image for one frame is obtained (S22: YES), the lesion-candidate-region evaluating section 133 performs processing for evaluating visibility of the respective plurality of lesion candidate regions L included in the endoscopic image for one frame (step S23 in FIG. 6).

More specifically, for example, the lesion-candidate-region evaluating section 133 calculates, based on the endoscopic image E2 and the lesion candidate information IL21, a contrast value CA equivalent to a value of a luminance ratio of the lesion candidate region L21 and a peripheral region of the lesion candidate region L21. For example, the lesion-candidate-region evaluating section 133 calculates, based on the endoscopic image E2 and the lesion candidate information a contrast value CB equivalent to a value of a luminance ratio of the lesion candidate region L22 and a peripheral region of the lesion candidate region L22. For example, the lesion-candidate-region evaluating section 133 calculates, based on the endoscopic image E2 and the lesion candidate information IL23, a contrast value CC equivalent to a value of a luminance ratio of the lesion candidate region L23 and a peripheral region of the lesion candidate region L23.

For example, the lesion-candidate-region evaluating section 133 compares the contrast value CA and predetermined thresholds THB and THC (it is assumed that THB<THC) to thereby evaluate visibility of the lesion candidate region L21. For example, when obtaining a comparison result indicating CA<THB, the lesion-candidate-region evaluating section 133 obtains an evaluation result indicating that the visibility of the lesion candidate region L21 is low. For example, when obtaining a comparison result indicating THB≤CA≤THC, the lesion-candidate-region evaluating section 133 obtains an evaluation result indicating that the visibility of the lesion candidate region L21 is a medium degree. For example, when obtaining a comparison result indicating THC<CA, the lesion-candidate-region evaluating section 133 obtains an evaluation result indicating that the visibility of the lesion candidate region L21 is high. Note that, in FIG. 7, an example is shown in which THC<CA, that is, the evaluation result indicating that the visibility of the lesion candidate region L21 is high is obtained.

For example, the lesion-candidate-region evaluating section 133 compares the contrast value CB and the predetermined thresholds THB and TUC to thereby evaluate visibility of the lesion candidate region L22. For example, when obtaining a comparison result indicating CB<THB, the lesion-candidate-region evaluating section 133 obtains an evaluation result indicating that the visibility of the lesion candidate region L22 is low. For example, when obtaining a comparison result indicating THB≤CB≤THC, the lesion-candidate-region evaluating section 133 obtains an evaluation result indicating that the visibility of the lesion candidate region L22 is a medium degree. For example, when obtaining a comparison result indicating THC<CB, the lesion-candidate-region evaluating section 133 obtains an evaluation result indicating that the visibility of the lesion candidate region L22 is high. Note that, in FIG. 7, an example is shown in which THB≤CB≤THC, that is, the evaluation result indicating that the visibility of the lesion candidate region L22 is a medium degree is obtained.

For example, the lesion-candidate-region evaluating section 133 compares the contrast value CC and the predetermined thresholds THB and THC to thereby evaluate visibility of the lesion candidate region L23. For example, when obtaining a comparison result indicating CC>THB, the lesion-candidate-region evaluating section 133 obtains an evaluation result indicating that the visibility of the lesion candidate region L23 is low. For example, when obtaining a comparison result. indicating THB≤CC≤THC, the lesion-candidate-region evaluating section 133 obtains an evaluation result indicating that the visibility of the lesion candidate region L23 is a medium degree. For example, when obtaining a comparison result indicating THC<CC, the lesion-candidate-region evaluating section 133 obtains an evaluation result indicating that the visibility of the lesion candidate region L23 is high. Note that, in FIG. 7, an example is shown in which THC<CC, that is, the evaluation result indicating that the visibility of the lesion candidate region L23 is low is obtained.

When a determination result indicating that a plurality of lesion candidate regions L are detected from the endoscopic image for one frame is obtained (S22: YES), the display control section 134 performs processing for setting, based on the evaluation result in step S23 in FIG. 6, the marker image M added by the highlighting processing of the highlighting processing section 134A (step S24 in FIG. 6).

More specifically, for example, the display control section 134 respectively sets, based on the evaluation result in step S23 in FIG. 6, a marker image M21 for highlighting, with a highlighting amount MA, a position of the lesion candidate region L21 having high visibility, a marker image M22 for highlighting, with a highlighting amount MB larger than the highlighting amount MA, a position of the lesion candidate region L22 having medium visibility, and a marker image M23 for highlighting, with a highlighting amount MC larger than the highlighting amount MB, a position of the lesion candidate region L23 having low visibility.

In other words, according to the processing in step S24 in FIG. 6, when an evaluation result indicating that visibility of one lesion candidate region among a plurality of lesion candidate regions detected from an endoscopic image for one frame is high is obtained, setting for relatively reducing a highlighting amount in highlighting a position of the one lesion candidate region is performed by the display control section 134. According to the processing in step S24 in FIG. 6, when an evaluation result indicating that visibility of one lesion candidate region among a plurality of lesion candidate regions detected from an endoscopic image for one frame is low is obtained, setting for relatively increasing a highlighting amount in highlighting a position of the one lesion candidate region is performed by the display control section 134.

When a determination result indicating that one lesion candidate region L is detected from the endoscopic image for one frame is obtained (S22: NO), the display control section 134 sets the marker image M for highlighting a position of the one lesion candidate region L (step S25 in FIG. 6). Note that, in the present embodiment, for example, the marker image M same as the marker image M22 may be set by the processing in step S25 in FIG. 6.

The highlighting processing section 134A performs processing for generating, based on the lesion candidate information IL obtained as the processing result of step S21 in FIG. 6, the marker image M set through the processing in step S24 or step S25 in FIG. 6 and adding the generated marker image M to the endoscopic image (step S26 in FIG. 6).

More specifically, for example, the highlighting processing section 134A generates, based on the lesion candidate information IL21, the marker image M21 set through the processing in step S24 in FIG. 6 and adds the generated marker image M21 to a periphery of the lesion candidate region L21 in the endoscopic image E2. According to such processing of the highlighting processing section 134A, for example, the marker image M21, which is a rectangular frame having a line width WA corresponding to the highlighting amount MA and surrounding the periphery of the lesion candidate region L21, is added to the endoscopic image E2.

For example, the highlighting processing section 134A generates, based on the lesion candidate information IL22, the marker image M22 set through the processing in step S24 in FIG. 6 and adds the generated marker image M22 to a periphery of the lesion candidate region L22 in the endoscopic image E2. According to such processing of the highlighting processing section 134A, for example, the marker image M22, which is a rectangular frame having a line width WB (>WA) corresponding to the highlighting amount MB and surrounding the periphery of the lesion candidate region L22, is added to the endoscopic image E2.

For example, the highlighting processing section 134A generates, based on the lesion candidate information IL23, the marker image M23 set through the processing in step S24 in FIG. 6 and adds the generated marker image M23 to a periphery of the lesion candidate region L23 in the endoscopic image E2. According to such processing of the highlighting processing section 134A, for example, the marker image M23, which is a rectangular frame having a line width WC (>WB) corresponding to the highlighting amount MC and surrounding the periphery of the lesion candidate region L23, is added to the endoscopic image E2.

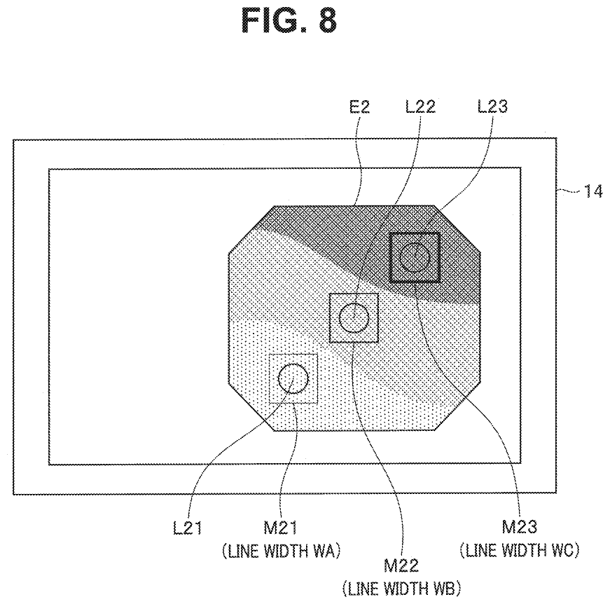

In other words, when the processing in step S26 is performed through the processing in step S24 in FIG. 6, a display image obtained by respectively adding, to the endoscopic image E2, the marker image M21 surrounding the periphery of the lesion candidate region L21 with a frame line having the line width WA, the marker image M22 surrounding the periphery of the lesion candidate region L22 with a frame line having the line width WB larger than the line width WA, and the marker image M23 surrounding the periphery of the lesion candidate region L23 with a frame line having the line width WC larger than the line width WB is generated. The generated display image is displayed on the display apparatus 14 (see FIG. 8). FIG. 8 is a diagram schematically showing an example of a display image displayed on the display apparatus through processing of the endoscopic image processing apparatus according to the second embodiment.

For example, the highlighting processing section 134A performs processing for generating, based on the lesion candidate information IL obtained as the processing result of step S21 in FIG. 6, the marker image M set through the processing in step S25 in FIG. 6 and adding the generated marker image M to a periphery of one lesion candidate region L in the endoscopic image. According to such processing of the highlighting processing section 134A, for example, a display image obtained by adding the marker image M (same as the marker image M22) surrounding a periphery of the lesion candidate region L to the endoscopic image E2 is generated. The generated display image is displayed on the display apparatus 14 (not illustrated).

As explained above, according to the present embodiment, when a plurality of lesion candidate regions are included in an endoscopic image, a marker image for highlighting, with a relatively large highlighting amount, a position of a lesion candidate region having low visibility and a marker image for highlighting, with a relatively small highlighting amount, a position of a lesion candidate region having high visibility can be respectively added to the endoscopic image. Therefore, according to the present embodiment, it is possible to, without obstructing visual recognition of a lesion candidate region included in the endoscopic image as much as possible, inform presence of the lesion candidate region.

Note that, according to the present embodiment, the processing performed in step S23 in FIG. 6 is not limited to the processing for evaluating, based on a contrast value of a lesion candidate region included in an endoscopic image, visibility of the lesion candidate region. For example, processing for evaluating the visibility of the lesion candidate region based on a size of the lesion candidate region may be performed. In such a case, for example, when the size of the lesion candidate region included in the endoscopic image is small, an evaluation result indicating that the visibility of the lesion candidate region is low is obtained. In the case explained above, for example, when the size of the lesion candidate region included in the endoscopic image is large, an evaluation result indicating that the visibility of the lesion candidate region is high is obtained.

According to the present embodiment, the processing performed in step S23 in FIG. 6 is not limited to the processing for evaluating, based on a contrast value of a lesion candidate region included in an endoscopic image, visibility of the lesion candidate region. For example, processing for evaluating the visibility of the lesion candidate region based on a spatial frequency component of the lesion candidate region may be performed. In such a case, for example, when the spatial frequency component of the lesion candidate region included in the endoscopic image is low, an evaluation result indicating that the visibility of the lesion candidate region is low is obtained. In the case explained above, for example, when the spatial frequency component of the lesion candidate region included in the endoscopic image is high, an evaluation result indicating that the visibility of the lesion candidate region is high is obtained.

In other words, according to the present embodiment, in step S23 in FIG. 6, processing for evaluating, based on any of a contrast value, a size, or a spatial. frequency component of one lesion candidate region among a plurality of lesion candidate regions included in an endoscopic image for one frame, the visibility of the one lesion candidate region may be performed.

In the present embodiment, according to an evaluation result of visibility of a plurality of lesion candidate regions, a display form of a plurality of marker images for highlighting positions of the respective plurality of lesion candidate regions may be changed. More specifically, in the present embodiment, for example, processing for changing, according to the evaluation result of the visibility of the plurality of lesion candidate regions, at least one of a line width, a hue, chroma, brightness, or a shape of frame lines of a plurality of marker images, which are frames surrounding peripheries of the respective plurality of lesion candidate regions, may be changed by the display control section 134.

### (Third Embodiment)

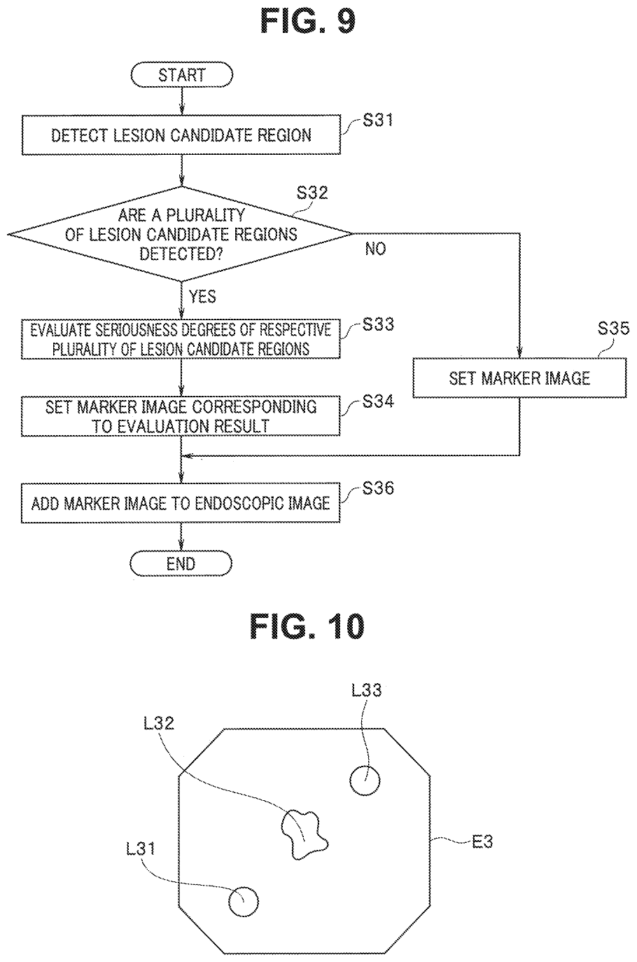

FIGS. 9 to 11 relate to a third embodiment of the present invention.

Note that, in the present embodiment, detailed explanation concerning portions having the same configurations and the like as the configurations and the like in at least one of the first or second embodiments is omitted. Portions having configurations and the like different from the configurations and the like in both of the first and second embodiments are mainly explained.

The endoscopic image processing apparatus 13 in the present embodiment is configured to perform processing different from the processing explained in the first and second embodiments. Specific examples of processing performed in sections of the endoscopic image processing apparatus 13 in the present embodiment are explained with reference to FIG. 9 and the like. FIG. 9 is a flowchart for explaining a specific example of processing performed in an endoscopic image processing apparatus according to the third embodiment.

The lesion-candidate-region detecting section 131 performs processing for detecting the lesion candidate region L included in an endoscopic image outputted from the main body apparatus 12 and performs processing for acquiring the lesion candidate information IL, which is information indicating the detected lesion candidate region L (step S31 in FIG. 9).

More specifically, according to the processing in step S31 in FIG. 9, for example, the lesion-candidate-region detecting section 131 detects three lesion candidate regions L31, L32, and L33 included in an endoscopic image E3 for one frame shown in FIG. 10 and respectively acquires lesion candidate information IL31 corresponding to the lesion candidate region L31, lesion candidate information IL32 corresponding to the lesion candidate region L32, and lesion candidate information IL33 corresponding to the lesion candidate region L33. In other words, in such a case, the lesion candidate regions L31, L32, and L33 and the lesion candidate information IL31, IL32, and IL33 are acquired as a processing result of step S31 in FIG. 9. FIG. 10 is a diagram schematically showing an example of an endoscopic image set as a processing target of the endoscopic image processing apparatus according to the third embodiment.

The determining section 132 performs processing for determining, based on the processing result of step S31 in FIG. 9, whether a plurality of lesion candidate regions L are detected from an endoscopic image for one frame (step S32 in FIG. 9).

When a determination result indicating that a plurality of lesion candidate regions L are detected from the endoscopic image for one frame is obtained (S32: YES), the lesion-candidate-region evaluating section 133 performs processing for evaluating seriousness degrees of the respective plurality of lesion candidate regions L included in the endoscopic image for one frame (step S33 in FIG. 9).

More specifically, for example, the lesion-candidate-region evaluating section 133 acquires, based on the endoscopic image E3 and the lesion candidate information IL31, a class CP equivalent to a classification result obtained by classifying the lesion candidate region L31 according to a predetermined classification standard CK having a plurality of classes for classifying lesions such as a polyp. The lesion-candidate-region evaluating section 133 acquires, based on the endoscopic image E3 and the lesion candidate information IL32, a class CQ equivalent to a classification result obtained by classifying the lesion candidate region L32 according to the predetermined classification standard CK. The lesion-candidate-region evaluating section 133 acquires, based on the endoscopic image E3 and the lesion candidate information IL33, a class CR equivalent to a classification result obtained by classifying the lesion candidate region L33 according to the predetermined classification standard CK. Note that, in the present embodiment, as the predetermined classification standard CK, for example, a classification standard with which a classification result corresponding to at least one of a shape, a size, or a color tone of a lesion candidate region can be obtained may be used.

The lesion-candidate-region evaluating section 133 evaluates a seriousness degree of the lesion candidate region L31 based on the class CP acquired as explained above and obtains an evaluation result. The lesion-candidate-region evaluating section 133 evaluates a seriousness degree of the lesion candidate region L32 based on the class CQ acquired as explained above and obtains an evaluation result. The lesion-candidate-region evaluating section 133 evaluates a seriousness degree of the lesion candidate region L33 based on the class CR acquired as explained above and obtains an evaluation result. Note that, in FIG. 10, an example is shown in which evaluation results in which the seriousness degrees of the lesion candidate regions L31 and L33 are substantially the same are obtained and evaluation results in which the seriousness degree of the lesion candidate region L32 is relatively higher than the seriousness degrees of the lesion candidate regions L31 and L33 are obtained.

When a determination result indicating that a plurality of lesion candidate regions L are detected from the endoscopic image for one frame is obtained (S32: YES), the display control section 134 performs processing for setting, based on an evaluation result of step S33 in FIG. 9, the marker image M added by the highlighting processing of the highlighting processing section 134A (step S34 in FIG. 9).

More specifically, for example, the display control section 134 sets, based on the evaluation result of step S33 in FIG. 9, a marker image M32 for highlighting a position of the lesion candidate region L32 having the highest seriousness degree among the lesion candidate regions L31, L32, and L33.

In other words, according to the processing in step S34 in FIG. 9, setting for highlighting a position of one lesion candidate region having the highest seriousness degree among a plurality of lesion candidate regions detected from an endoscopic image for one frame is performed by the display control section 134.

When a determination result indicating that one lesion candidate region L is detected from the endoscopic image for one frame is obtained (S32, NO), the display control section 134 sets the marker image M for highlighting a position of the one lesion candidate region L (step S35 in FIG. 9). Note that, in the present embodiment, for example, the marker image M same as the marker image M32 explained above may be set by the processing in step S35 in FIG. 9.

The highlighting processing section 134A performs processing for generating, based on the lesion candidate information IL obtained as the processing result of the step S31 in FIG. 9, the marker image M set through the processing in step S34 or step S35 in FIG. 9 and adding the generated marker image M to the endoscopic image (step S36 in FIG. 9).

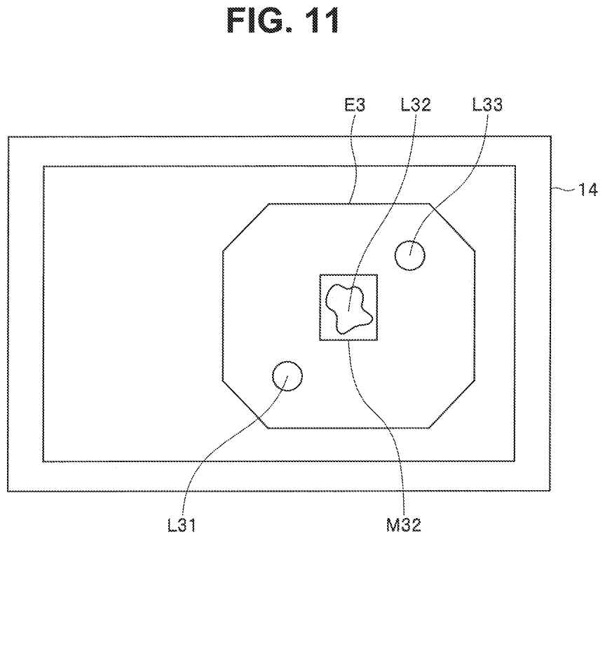

More specifically, for example, the highlighting processing section 134A performs processing for generating, based on the lesion candidate information IL32, the marker image M32 set through the processing in step S34 in FIG. 9 and adding the generated marker image M32 to the lesion candidate region L32 in the endoscopic image E3. According to such processing of the highlighting processing section 134A, for example, a display image obtained by adding the marker image M32, which is a rectangular frame surrounding a periphery of the lesion candidate region L32, to the endoscopic image E3 is generated. The generated display image is displayed on the display apparatus 14 (see FIG. 11). FIG. 11 is a diagram schematically showing an example of a display image displayed on a display apparatus through processing of the endoscopic image processing apparatus according to the third embodiment.

For example, the highlighting processing section 134A performs processing for generating, based on the lesion candidate information IL Obtained as the processing result of step S31 in FIG. 9, the marker image M set through the processing in step S35 in FIG. 9 and adding the generated marker image M to a periphery of one lesion candidate region L in the endoscopic image. According to such processing of the highlighting processing section 134A, for example, a display image obtained by adding the marker image M (same as the marker image M32) surrounding the periphery of the lesion candidate region L to the endoscopic image E3 is generated. The generated display image is displayed on the display apparatus 14 (not illustrated).

As explained above, according to the present embodiment, only a position of one lesion candidate region having the highest seriousness degree among a plurality of lesion candidate regions included in an endoscopic image can be highlighted. In other words, according to the present embodiment, when a plurality of lesion candidate regions are included in an endoscopic image, it is possible to add a marker image for highlighting a position of a lesion candidate region having a high seriousness degree to the endoscopic image without adding a marker image for highlighting a position of a lesion candidate region having low seriousness degree to the endoscopic image. Therefore, according to the present embodiment, it is possible to, without obstructing visual recognition of a lesion candidate region included in the endoscopic image as much as possible, inform presence of the lesion candidate region.

Note that, according to the present embodiment, a marker image added to an endoscopic image is not limited to a marker image for highlighting a position of one lesion candidate region having the highest seriousness degree among a plurality of lesion candidate regions included in the endoscopic image. For example, a marker image for highlighting positions of one or more lesion candidate regions classified into a high seriousness degree class in the predetermined classification standard CK may be added to the endoscopic image. In other words, according to the present embodiment, setting for highlighting positions of one or more lesion candidate regions classified into a high seriousness degree class in the predetermined classification standard CK among a plurality of lesion candidate regions detected from an endoscopic image for one frame may be performed by the display control section 134. In such a case, for example, when a plurality of lesion candidate regions classified into the high seriousness degree class in the predetermined classification standard CK are included in an endoscopic image, a plurality of marker images for highlighting the positions of the respective plurality of lesion candidate regions are added to the endoscopic image.

The present invention is not limited to the embodiments explained above. It goes without saying that various changes and applications are possible within a range not departing from the gist of the invention.

What is claimed is:

1. An endoscopic image processing apparatus comprising a processor, wherein
the processor
detects a lesion candidate region included in an endoscopic image obtained by picking up an image of an inside of a. subject with an endoscope,
highlights a position of the lesion candidate region detected from the endoscopic image,
when a plurality of lesion candidate regions are detected from the endoscopic image, relatively evaluates visibility of the plurality of lesion candidate regions, and
performs setting for position highlighting of the lesion candidate region based on an evaluation result of the visibility.

2. The endoscopic image processing apparatus according to claim 1, wherein the processor evaluates, based on any of a contrast value, a size, or a spatial frequency component of one lesion candidate region among the plurality of lesion candidate regions, the visibility of the one lesion. candidate region.

3. The endoscopic image processing apparatus according to claim 1, wherein
when the evaluation result indicating that the visibility of the lesion candidate region is high is obtained, the processor relatively reduces a highlighting amount in highlighting the position of the lesion candidate region, and
when the evaluation result indicating that the visibility of the lesion candidate region is low is obtained, the processor relatively increases the highlighting amount in highlighting the position of the lesion candidate region.

4. The endoscopic image processing apparatus according to claim 1, wherein the processor adds, to the endoscopic image, a plurality of marker images, which are frames surrounding peripheries of the respective plurality of lesion candidate regions, and changes at least one of a line width, a hue, chroma, brightness, or a shape of frame lines of the plurality of marker images according to the evaluation result of the visibility of the respective plurality of lesion candidate regions.

5. An endoscopic image processing method comprising:
detecting a lesion candidate region included in an endoscopic image obtained by picking up an image of an inside of a subject with an endoscope;
highlighting a position of the lesion candidate region detected from the endoscopic image;
when a plurality of lesion candidate regions are detected from the endoscopic image, relatively evaluating visibility of the plurality of lesion candidate regions; and
performing setting for position highlighting of the lesion candidate region based on an evaluation result of the visibility.

6. A computer-readable non-transitory recording medium that stores a program,
the program causing a computer to:
detect a lesion candidate region included in an endoscopic image obtained by picking up an image of an inside of a subject with an endoscope;
highlight a position of the lesion candidate region detected from the endoscopic image;
when a plurality of lesion candidate regions are detected from the endoscopic image, relatively evaluate visibility of the plurality of lesion candidate regions; and
perform setting for position highlighting of the lesion candidate region based on an evaluation result of the visibility.
